(19) United States
(12) Patent Application Publication
(10) Pub. No.: US 2010/0311092 A1
(43) Pub. Date: Dec. 9, 2010

(54) METABOLIC FUEL SWITCHING BIOMARKER

(76) Inventor: Irwin J. Kurland, Stoney Brook, NY (US)

Correspondence Address: THE FARRELL LAW FIRM, LLP, 290 Broadhollow Road, Suite 210E, Melville, NY, 11747, US

(21) Appl. No.: 12/745,364

(22) PCT Filed: Dec. 1, 2008
(86) PCT No.: PCT/US08/85187

§ 371 Date: May 28, 2010

Related U.S. Application Data
(60) Provisional application No. 60/991,418, filed on Nov. 30, 2007.

(51) Int. Cl. C12Q 1/54; G01N 33/00

(52) U.S. Cl. 435/14; 436/95

(57) ABSTRACT

Disclosed is a method for measuring and determining whole body insulin release functionality, in which a plurality of physiologically acceptable differentially labeled carbohydrates is administered. A first administered carbohydrate label metabolizes faster than at least one of the other administered carbohydrate labels and recycling rates of at least two of the plurality of labeled carbohydrates are monitored. First and subsequent insulin release phases are detected. The first insulin release phase is detected by comparison of a recycling rate of the first administered carbohydrate with a recycling rate of the subsequent administered carbohydrate.

### PRIORITY

This application claims priority to provisional application Ser. No. 60/991,418, filed with the U.S. Patent and Trademark Office on Nov. 30, 2007, the contents of which is incorporated herein by reference.

### GOVERNMENT SPONSORSHIP

This invention was made with government support under grant number CA111577 awarded by the National Institutes of Health. The government has certain rights in the invention.

### BACKGROUND OF THE INVENTION

Obesity and diabetes are becoming epidemic, particularly in Western nations. Diabetes is a chronic condition involving high blood glucose resulting from various enzymatic/metabolic disorders involving muscle, fat, islet cell and the liver. Detection of a pre-diabetic state, or a propensity towards the development of diabetes, would enable early intervention and treatment. However, the physiological state that precedes diabetes onset is a subtle disorder lacking a well-defined diagnostic criterion. See, Definition and Diagnosis of Diabetes Mellitus and Intermediate Hyperglycemia, Report of a Consultation of World Health Organization/International Diabetes Federation, http://www.who.int/diabetes/publications/en/ (ISBN 978 92 4 159493 6) (2006).

The pre-diabetic state is generally a state other than an impaired glucose tolerance condition, defined either from fasting, and/or 2 hours blood glucose values, or a ‘metabolic syndrome.’ The metabolic syndrome is another attempt to define a pre-disposition to diabetes, and is based on a number of criteria found by examining body habitus, i.e. degree of obesity, and glucose/lipid profiles in selected patient populations. As shown in FIGS. 2(a) and 2(b), metabolic flexibility differs in lean and obese, insulin resistant individuals, during fasting and during insulin-stimulated conditions.

Metabolic flexibility is generally the ability of an organism to ‘switch’ from using fats in the bloodstream, i.e. fatty acids, when in the fasted state, to glucose after meals. It is a normal physiological reaction to use the fuel that exists in excess for energy. This, in part, is due to the action of insulin. Insulin is low in the fasted state, and the low insulin levels, together with other hormones that are high in the fasted state, i.e. ‘counter-regulatory’ hormones, promote the metabolism of fats. When glucose is high after meals, insulin levels are high, the counter-regulatory hormone levels are low, and glucose disposal increases. Obese, ‘pre-diabetic’ individuals, who technically may not have diabetes or an impaired glucose tolerance, can be shown to have an impairment in normal metabolic fuel (fats and carbohydrates) handling, even if such individuals are able to secrete elevated levels of insulin to maintain glucose tolerance. However, such showing requires laborious methods that are not suitable to widespread population evaluation. See WHO/IDF Report of a Consultation, supra. Such insulin resistant individuals, who are additionally termed metabolically inflexible, even if they satisfy the present criteria for normal glucose tolerance in fasted and fed states, may inappropriately over-utilize glucose as a fuel in the fasted state or may inappropriately over-utilize fatty acids as a fuel after consuming carbohydrates.

Conventional criterion for prediction of the pre-diabetic state fails to distinguish different genders and different ethnic subpopulations that typically have characteristics of the metabolic syndrome for greater or lesser degrees of obesity. Regardless of whether the problems are discussed using definitions of an impaired glucose tolerance or the metabolic syndrome, a need exists for a uniform method to detect problems in metabolic fuel handling.

Problems with metabolic fuel handling often signal the imminent, or progressive, onset of the pre-diabetic state. Current modes of diagnosing these metabolic disorders by the standard glucose tolerance test cannot accurately assess the dynamic deregulation in hepatic peripheral glucose disposal and peripheral fatty acid metabolism. Peripheral, i.e. mainly skeletal muscle, insulin resistance can be defined as the failure to adequately dispose, i.e. metabolize, glucose in response to glucose and insulin elevations seen after a meal. Metabolic flexibility is a broader definition, as it examines the ability not only to metabolize glucose, or fatty acids, but to ‘switch’ between uses of glucose and fatty acids dynamically, for example, hepatic insulin resistance, i.e. the failure to restrain glucose output or increased glucose disposal in response to glucose and insulin elevations, seen after a meal.

Two major forms of glucose tolerance testing are known to exist: an Oral Glucose Tolerance Test (OGTT) and an Intravenous Glucose Tolerance Test (IVGTT), given with and without a stable glucose label. However, neither test can assess a degree of glucose recycling through the liver, nor can either test yield a term indicative of hepatic insulin sensitivity, and the conventional tests fail to assess fatty acid usage.

That is, conventional tests measure only a decrease in peripheral plasma insulin. Using mathematical modeling, conventional systems predict portal insulin secretion rates. However, conventional methods fail to examine efficacy of first phase insulin release, which the present invention recognizes as crucial for ‘switching’ the liver from glucose production in the fasting state to glucose metabolizing in the fed state.

The present invention overcomes the problems of conventional methods and apparatus used to diagnose the pre-diabetic state, by providing an easily administered test to assess in vivo metabolic flexibility, i.e. the ability of an individual to switch between usage of fatty acids primarily as fuel in the fasted state, to glucose primarily as fuel in the fed state. The present invention assesses tissue specific defects in metabolic flexibility in response to consuming glucose, for improved classification and detection of the pre-diabetic state.

### SUMMARY OF THE INVENTION

The present invention overcomes the above-described shortcomings of conventional systems by providing an apparatus and method for measuring and determining whole body insulin release functionality by monitoring first and subsequent insulin release phases in a linear fashion, preferably by administering a plurality of physiologically acceptable differentially labeled carbohydrates and detecting whether a first administered carbohydrate label metabolizes faster than at least one of the administered carbohydrate labels by monitoring recycling rates of at least two of the plurality of labeled carbohydrates.

A preferred embodiment of the present invention provides an expedited assessment of an individual's ability to switch between fuel sources, e.g. between fats and glucose stored in the bloodstream. In a preferred embodiment, a mass spectrometric assessment is performed of glucose and fatty acids metabolites excreted by the individual following ingestion of labeled glucose.

In a preferred embodiment of the present invention, glucose cycling is utilized to detect disturbances in hepatic insulin resistance, providing a more sensitive cycling detection than obtained by conventional measurement of glucose production.

The present invention provides a method and apparatus for measuring hepatic and peripheral response to a first phase insulin release, preferably by tracking recycling rates of stable isotopes, preferably [6,6-²H₂] glucose and [2-²H₁] glucose as labeled carbohydrates whose metabolites are measured at intervals and analyzed over time via mass spectrometry. In a preferred embodiment, detected dynamic changes in plasma metabolites reflect a balance between whole body glucose utilization and uptake and recirculation of glucose by the liver such that peripheral glucose utilization results in the equal disposal of [6,6-²H₂] glucose and [2-²H₁] glucose, and hepatic glucose recycling results in the disappearance of the [2-²H₁] glucose relative to the [6,6-²H₂] glucose. In a preferred embodiment, the level of hepatic glucose recycling and peripheral glucose utilization determined by whole body dynamic changes in plasma metabolites reflect intactness of the recycling of glucose by the subject's liver, and glucose utilization in the body's peripheral tissues, i.e. skeletal muscle, and is a sensitive measure of the functionality of first phase insulin release reflecting the degree of compensatory interactions between the liver and periphery.

In a preferred embodiment of the present invention, a Dynamic Assessment of Fuel Switching Test (DAFST) is provided that uses stable isotopes to estimate a degree of hepatic glucose uptake and recycling. The DAFST of the present invention assesses increases in glucose uptake by the liver by a first phase insulin release, as well as the combined effect of first and second phase insulin release on glucose disposal. The DAFST of the present invention also assesses whether metabolism of glucose or fatty acids are being effectively switched from primarily fatty acids in the fasted state to glucose in the fed state, and a level of effectiveness in overall conversion of fuel to the basic unit of acetyl CoA, which can come from glucose or fatty acids or amino acids in case of protein breakdown.

A preferred embodiment of the present invention enables preventive health studies to determine prevalence of the pre-diabetic state within different ethnic populations, preferably by age and gender.

A preferred embodiment of the present invention provides a method for screening and estimating probable onset of Type II Diabetes Mellitus (T2DM) in healthy appearing individuals and determination of optimal lifestyle/pharmagenomic mix of therapeutic agents to forestall development of T2DM in such individuals.

A preferred embodiment of the present invention identifies women likely to develop gestational diabetes, by administration of the DAFST of the present invention in the first trimester, preferably between six and twelve weeks from conception.

In a preferred embodiment of the present invention, a patient ingests glucose that includes first labeled glucose and second labeled glucose, and plasma sampled from the patient is analyzed to determine a relative level of preservation of the first or second labeled glucose. Selective recycling by the patient's liver and muscle (whole body) alters the relative level of preservation, and the first and second labeled glucoses preferably are [6,6-²H₂] glucose and [2-²H₁] glucose. In a preferred embodiment, levels of blood glucose and blood insulin are measured at predetermined intervals, along with mass spectrometric measurements of labeled glucoses and other fuels, and a curve is developed, the time dependence of which predicts rates of glucose recycling, or rates of fatty acid and other fuel utilizations. In a preferred embodiment of the present invention, an area under the curve is calculated based on the measurements, and diabetes onset is detected based on time course dependence of the labeled glucoses, other fuels, and/or the calculated area. In a preferred embodiment, the time course dependence of labeled glucoses and/or fuels, and/or calculated areas of these quantities indicates the patient's ability to dynamically switch fuels between glucose and fatty acids. In the present invention, the labeled glucose is non-toxic and the patient is a mammal. Gestational diabetes can be predicted by preferably performing the method of the present invention in a first or second trimester of pregnancy.

### BRIEF DESCRIPTION OF THE DRAWINGS

The above and other objects, features and advantages of certain exemplary embodiments of the present invention will be more apparent from the following detailed description taken in conjunction with the accompanying drawings, in which:

FIGS. 1(a)-(b) show first phase insulin release and total insulin release, predicted from the fasting state, showing that first phase insulin release and total insulin release can be predicted from the fasting state, and that total plasma insulin Area Under the Curve (AUC) peaks and then declines as an Impaired Fasting Glucose (IFG) range is reached;

FIGS. 2(a)-(b) compare summaries of metabolic flexibility of lean and obese, insulin resistant individuals, during fasting and during insulin-stimulated conditions;

FIG. 3 shows results of a lack of first phase insulin release can result in postprandial hyperglycemia;

FIG. 4 shows pancreatic beta-cell failure to compensate for insulin resistance results in the progression of a pre-diabetic state/metabolic syndrome to T2DM;

FIG. 5(a) shows variation in plasma glucose over time following an insulin injection;

FIG. 5(b) shows variation in plasma glucose over time following a glucose injection;

FIG. 5(c) shows variation in plasma insulin over time following a glucose injection;

FIG. 5(d) shows variation in plasma lactate over time following a glucose injection;

FIG. 6(a) illustrates decay of [6,6-²H₂] glucose in a mouse model, reflecting mainly enhanced muscle uptake of glucose during a glucose tolerance test;

FIG. 6(b) illustrates a relative rate of de-deuteration in plasma of [6,6-²H₂] glucose compared to [2-²H₁] glucose over time after glucose injection;

FIG. 6(c) illustrates disappearance of [6,6-²H₂] glucose compared to [2-²H₁] glucose;

FIG. 7(a) illustrates hepatic glucose production as being the same in the fasted state of the Pten heterodeficient and wild type mice, despite increased hepatic insulin sensitivity for the Pten heterodeficient mice;

FIG. 7(b) illustrates a suppression of glucokinase in Pten heterodeficient mice (causing suppression of glucose recycling, see FIG. 6(b)) that underlies the mechanism for matching Hepatic Glucose Production (HGP), despite increased hepatic insulin sensitivity for the Pten heterodeficient mice;

FIG. 8 shows blood glucose and insulin variation for two human subjects in response to a 100 gram during an oral hepatic recycling deuterated glucose tolerance test (FIR-dGTT);

FIG. 9 shows a time course of hepatic glucose uptake and recycling for the second human subject over an entire GTT;

FIG. 10 shows hepatic glucose uptake and recycling for the second human subject early in the GTT (left panel) and late in the GTT where recycling is increased (right panel), attributed to the induction of glucokinase by first phase insulin release;

FIG. 11 shows hepatic glucose uptake and recycling for the first human subject (who has absent first phase insulin release, IFG and IGT) fixed throughout the OTT;

FIGS. 12(a)-(b) chart plasma acetyl-carnitine (C2) and plasma palmitoyl-carnitine (C16) measured during an oral HR-dGTT for subject 1;

FIGS. 13(a)-(b) chart C2 and C16 measured during an HR-dGTT for subject 2;

FIGS. 14(a)-(b) show a time course of D2 glucose ([6,6-²H₂] glucose) during the HR-dGTT for human subjects 1 and 2, respectively, reflecting predominately peripheral (skeletal muscle) glucose disposal;

FIG. 15(a) shows plasma glucose response to a 100 gram HR-dGTT in a pregnant patient at the end of the first trimester, and early third trimester;

FIG. 15(b) shows plasma insulin response to a 100 gram HR-dGTT in a pregnant patient at the end of the first trimester, and early third trimester;

FIG. 15(c) shows plasma D2 glucose response to a 100 gram HR-dGTT in a pregnant patient at the end of the first trimester, and early third trimester;

FIG. 15(d) shows plasma glucose recycling (1-D1/D2) response to a 100 gram HR-dGTT in a pregnant patient at the end of the first trimester, and early third trimester;

FIG. 15(e) shows the plasma GLP-1 response (a determinant/modulator of glucose's ability to stimulate insulin release) to a 100 gram HR-dGTT in a pregnant patient at the end of the first trimester, and early third trimester; and

FIG. 16 shows the blood leptin variation for the two human subjects of FIG. 8.

### DETAILED DESCRIPTION OF THE PREFERRED EMBODIMENTS

The following detailed description of preferred embodiments of the invention will be made in reference to the accompanying drawings. In describing the invention, explanation about related functions or constructions known in the art are omitted for the sake of clarity in understanding the concept of the invention, to avoid obscuring the invention with unnecessary detail.

The present invention assesses metabolic flexibility of an individual, i.e. the ability of an individual to switch between usage of fatty acids primarily as fuel in the fasted state, to glucose primarily as fuel in the fed state. That is, the DAFTS provided by the present invention assesses the metabolic flexibility of an individual. The DAFST predicts which individuals are predisposed towards the development of diabetes, and predicts whether the propensity may be due to hepatic versus skeletal muscle derangements in glucose handling, thereby facilitating optimal selection of drug agent(s) tailored for an individual's particular needs. The present invention is not limited to humans and can be adapted to various mammals, and is useful to detect drug efficacy, particularly in distinguishing between ethnic populations and gender and age variation. The present invention is not limited to comparing [6,6-²H₂] glucose to [2-²H₁] glucose, as other stable isotopes may be used, including but not limited to [1,2-¹³C] glucose and [1,6-¹³C] glucose.

Due to the stable isotopes utilized in the DAFST, the present invention provides more information than conventional glucose tolerance tests. The present invention provides plasma assessment of glucose and fatty acid metabolites using unique Gas Chromatography/Mass Spectrometry (GC/MS), as well as Liquid Chromatography/Mass Spectrometry (LC/MS) methodologies.

In a preferred embodiment, the DAFST assesses defects in metabolic fuel switching between glucose and fatty acids. The DAFST also assesses defects in hepatic glucose disposal and recycling, and peripheral glucose disposal during a deuterated glucose tolerance test, reflective of hepatic and peripheral insulin sensitivity. The DAFST assesses the metabolic flexibility via hormone measurements and mass spectrometric assessment of glucose and fatty acid metabolites that occur during a glucose tolerance test. Hormones measured in preferred embodiments of the present invention include, but are not limited to, insulin, glucagons, GLP-1 and GIP, with insulin being the most important hormone with regard to switching from fasted to fed states, and glucagon being important in regulating hepatic glucose production. GLP-1 (glucagon-like peptide 1) and GIP (gastric inhibitory peptide) are incretins secreted by the gut in response to food. GLP-1 and GIP boost first phase insulin resistance and decreased, or have decreased action, in patients who are obese and have T2DM, and with GLP-1 being a stronger, more clinically relevant factor than GIP, with GLP-1 forming a basis for a drug Byetta, used to help first phase insulin release disorders.

The DAFST addresses the issue of how to assess metabolic flexibility in a simple and consistent way. In a preferred embodiment, the DAFST employs two different mass spectrometric assessments of glucose and fatty acid utilization in response to the fasting state, and an oral glucose load. Glucose utilization is examined by administering a given glucose load, which, for example, can be 75 or 100 grams. For a DAFST using 100 grams of glucose, the test contains 10 grams of glucose that has a deuterium atom on the second carbon [2-²H₁] glucose, and 10 grams of glucose that has 2 deuterium atoms on the sixth carbon [6,6-²H₂] glucose, and 80 grams of unlabeled glucose. A preferred mass spectrometer is a GC/MS having the ability to separate out glucose molecules from other metabolites in plasma (gas chromatography) and then to examine the molecular fragments of glucose in the mass spectrometer to determine a ratio of [2-²H₁] glucose to [6,6-²H₂] glucose, or other stable glucose labels/isotopes that may be utilized in plasma.

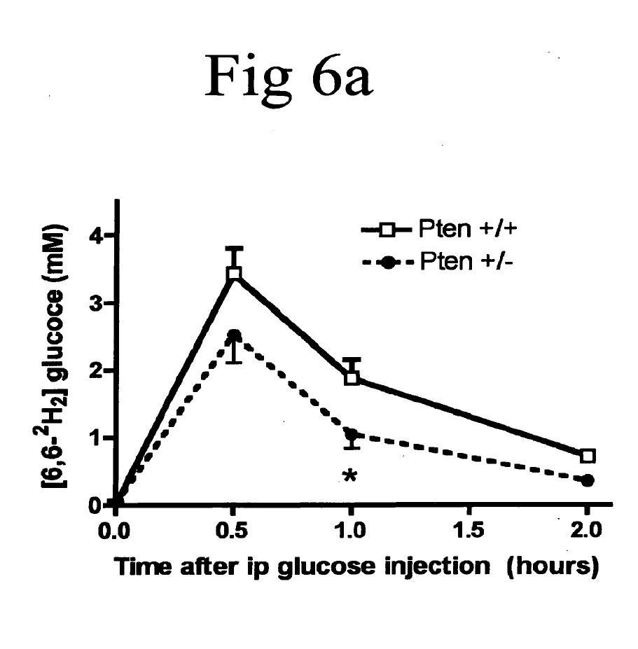

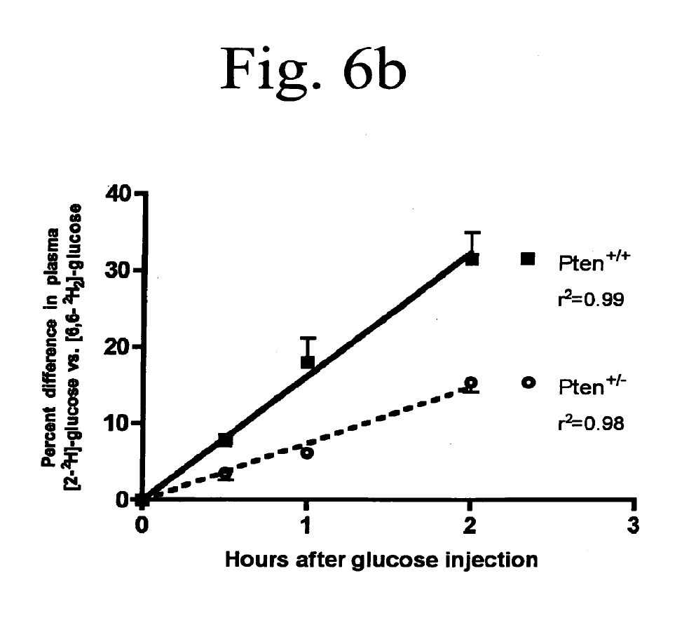

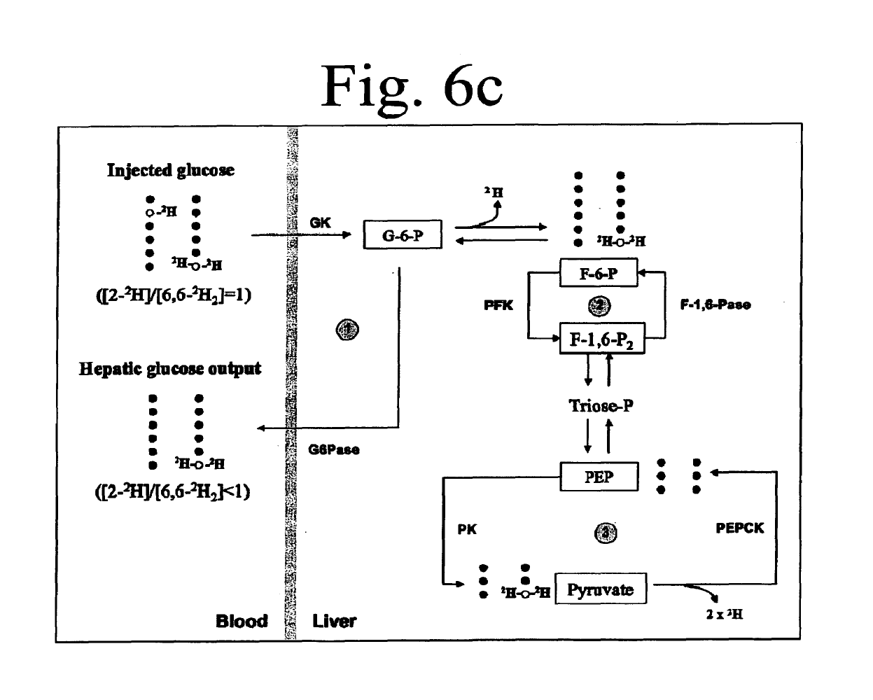

A difference between a disappearance rate from plasma of [2-²H₁] glucose vs. [6,6-²H₂] glucose reflects the differences between whole-body disposal of glucose ([6,6-²H₂] glucose disposal), and a degree of uptake and recirculation of glucose from the bloodstream of [2-²H₁] glucose by gluconeogenic tissues, such as the liver and kidney, with the liver being the overwhelming contributor under normal diurnal feeding conditions. As illustrated in FIG. 6(a)-6(c), differences in the disappearance rate from plasma of [2-²H₁] glucose vs. [6,6-²H₂] glucose reflect (mainly) liver disposal/exchange of [2-²H₁] glucose for unlabeled glucose and its export, diluting plasma [2-²H₁] glucose in comparison to [6,6-²H₂] glucose, as [6,6-²H₂] glucose and does not significantly exchange with unlabeled glucose in comparison to the rate that [2-²H₁] glucose does.

Examination of the formation of singly labeled [6-²H₁] glucose during glucose tolerance test studies determined that [6,6-²H₂] glucose did not significantly re-circulate. The singly labeled [6-²H₁] glucose theoretically could be produced by recirculation of [6,6-²H₂] glucose. See, Jun Xu et al., Decreased Hepatic Futile Cycling Compensates For Increased Glucose Disposal in the Pten Heterodeficient Mouse, Diabetes, Vol. 55, December 2006:3372-80, the contents of which is incorporated herein by reference. Accordingly, an AUC reflects whole-body disposal of [6,6-²H₂] glucose during the glucose tolerance test.

When [2-²H₁] glucose and [6,6-²H₁] glucose are administered as a 1:1 mixture, the disappearance of the two isotopes [2-²H₁] glucose and [6,6-²H₂] glucose can be determined by assessing the Carbon 1 to Carbon 4 fragment for [2-²H₁] glucose and the Carbon 3 to Carbon 6 fragment for [6,6-²H₂] glucose of the election-impact mass spectrometry on a GC/MS. In the present invention, the difference between the disappearance rates of [2-²H₁] glucose vs. [6,6-²H₂] glucose is recognized as the standard measure of hepatic futile cycling, i.e., the liver taking up glucose, converting that glucose to glucose-6-phosphate and then releasing it back to the blood stream as glucose, or converting that glucose to glycogen, depending upon what the body requires at the time.

The percent difference between a fraction of glucose molecules in plasma (the enrichments) of the two tracers reflects the relative rate of de-deuteration of [2-²H₁] vs. [6,6-²H₂] glucose, which is a measure of net hepatic glucose phosphorylation or, equivalently, glucose/glucose-6-P futile cycling.

The de-deuteration of [2-²H₁] glucose occurs when [2-²H₁] glucose becomes hepatic [2-²H₁] glucose-6-P and equilibration with fructose-6-P (FIG. 6(c)). Accordingly, the relative rate of de-deuteration of [2-²H₁] vs. [6,6-²H₂] glucose reflects hepatic glucose phosphorylation, as well as recycling, as the de-deuteration of [2-²H₁] glucose cannot occur unless glucose phosphorylation first occurs. The deuterium is lost in the equilibration between glucose-6-P and fructose-6-P, which occurs at a rate approximately 100 times faster than other reactions in the pathway. Hepatic [6,6-²H₂] glucose loses its deuterium after glycolysis, in the TCA cycle, when 3-carbon pyruvate produced is converted to oxaloacetate and when malate is converted to fumarate, a slower process at recycling steps much farther down the glycolytic pathway, than the glucose/glucose-6-P cycle where de-deuteration of [2-²H₁] glucose occurs. Therefore, if there is a substantial hepatic glucose uptake, plasma [2-²H₁] glucose concentration (enrichment) drops faster than plasma [6,6-²H₂] glucose enrichment.

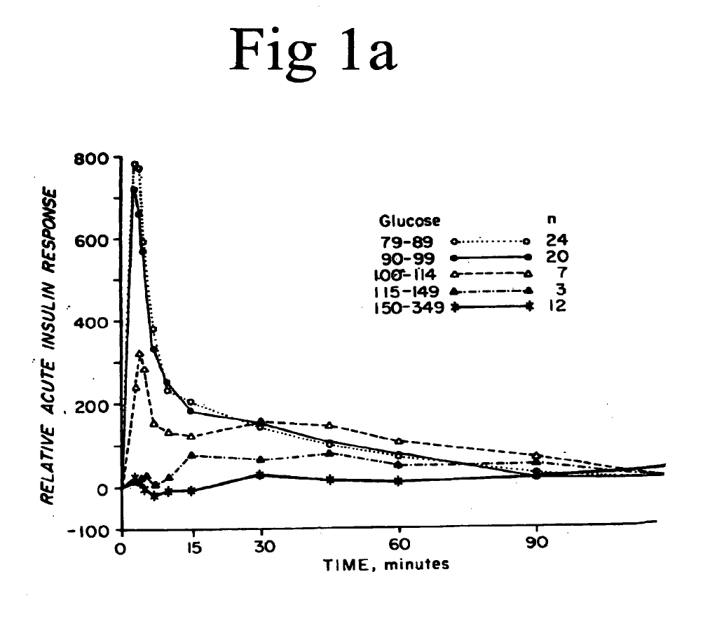

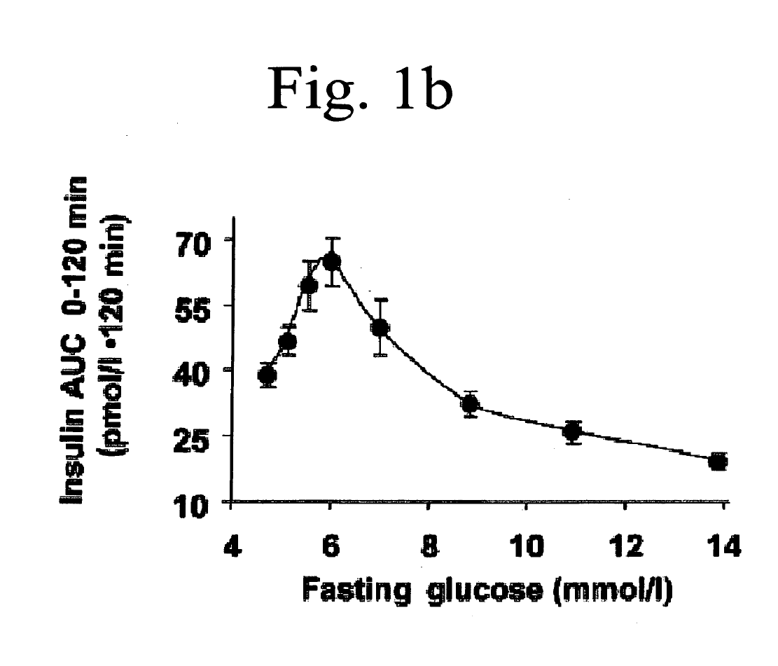

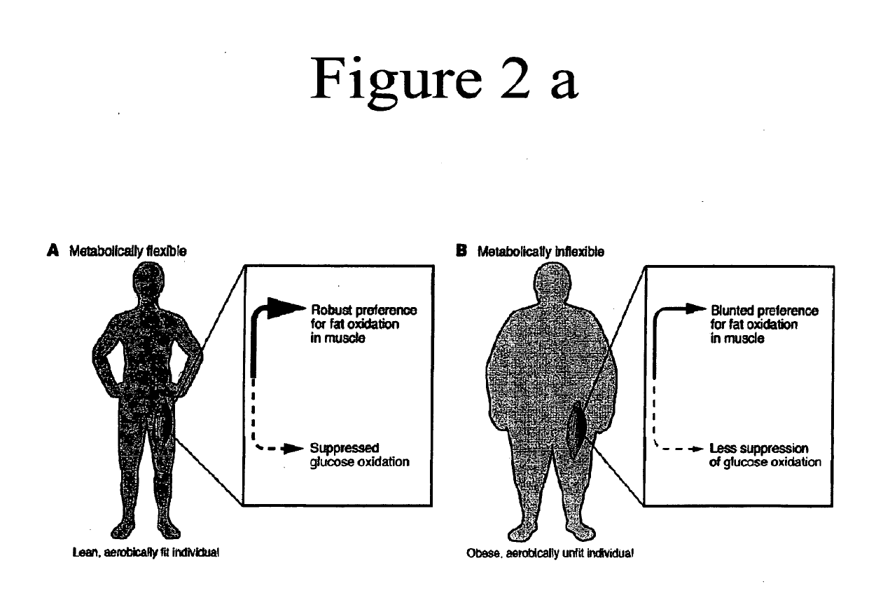

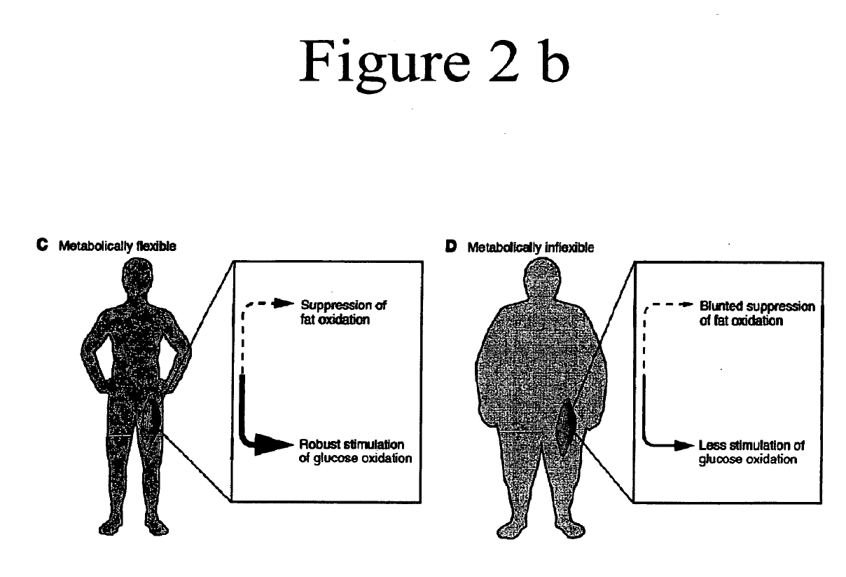

FIGS. 1(a) and 1(b) show first phase insulin release and total insulin release, predicted from the fasting glucose values. In FIG. 1(a), a first phase insulin release decreases as an IFG range is reached, for example at 100-114 mg/dl glucose. FIG. 1(b) shows the integration of the total AUC for both first and second phase insulin release. As first phase insulin release decreases, or is absent, second phase increases in compensation to lower (eventually) hyperglycemia, and FIG. 1(b) reflects the eventual failure of the compensatory mechanisms at plasma glucose levels beyond 6 milli-molar (mM). As shown in FIG. 1(a), first phase insulin release, as elicited by IVGTT, begins to decrease when fasting glucose reaches a 90-99 mg/dl range, and drops to approximately 50% for the IFG group of 110-114 mg/dl, compared to a 79-89 mg/dl Fasting Glucose (FG) group. Above 115 mg/dl, there is no appreciable amount of first phase insulin release. As background, FIG. 1(b) shows results from a study where the AUC elicited by a standard OGTT peaked at an FG defining the start of the IFG range, and declined thereafter.

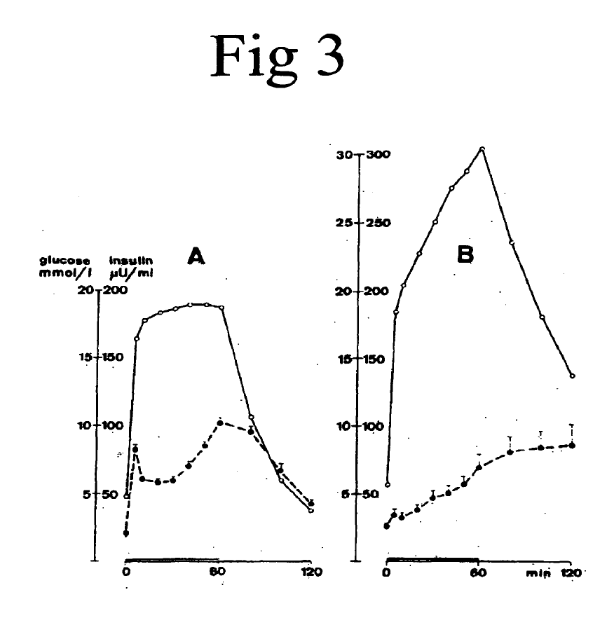

Regarding glucose intolerance, one of the earliest problems with insulin dynamics is a lack of recognition of the relevance of first-phase insulin secretion in response to a glucose stimulus, and use of the first-phase insulin secretion as a predictor of fuel homeostasis by the overall system. Lack of first phase insulin release results in post-prandial hyperglycemia, as shown in FIGS. 3(a) and 3(b), with the initial peak shown in dashed lines in FIG. 3(a) generally defining the first phase.

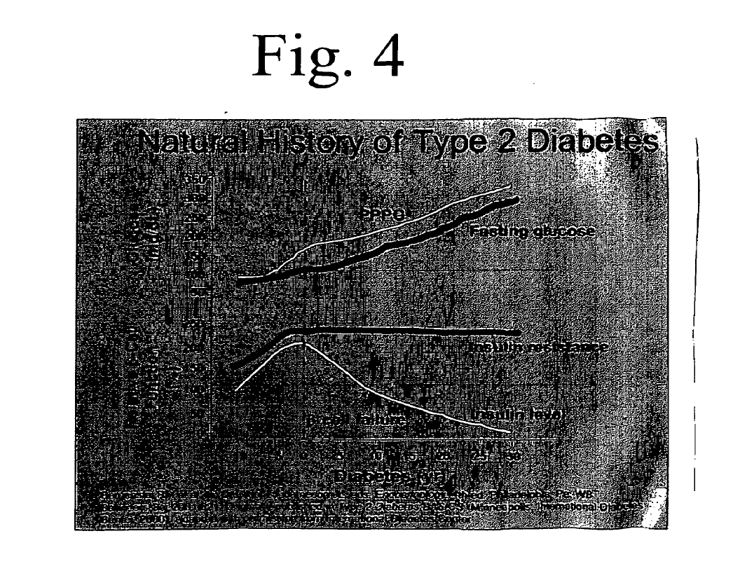

FIG. 4 shows failure of pancreatic beta-cells to compensate for insulin resistance results in the progression of a pre-diabetic state/metabolic syndrome to T2DM, with PPPG referring to post-prandial plasma glucose, as reported by Bergenstal E M et al., Endocrinology, 4^{th} Ed (2001). In the natural history of T2DM, there is observed an inverted U-shape of insulin secretion during progression from a state of normal glucose tolerance to T2DM (FIGS. 1(b) and 4). Fasting plasma insulin levels rise as fasting glucose levels climb into the range of impaired glucose tolerance. However, the fasting plasma insulin levels fall as diabetes worsens. As recognized in the present invention, rising insulin levels reflect a compensatory response to insulin resistance, while the falling levels are indicative of beta-cell failure, through a combination of impaired function of individual beta-cells, and the loss of beta-cell mass.

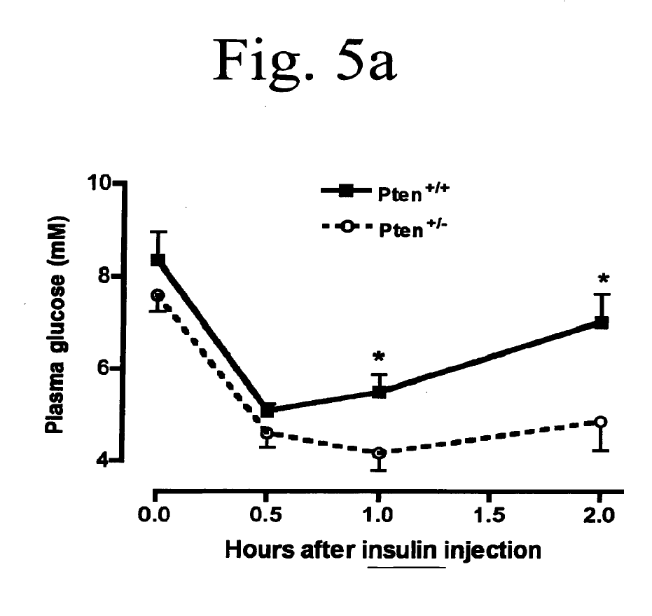

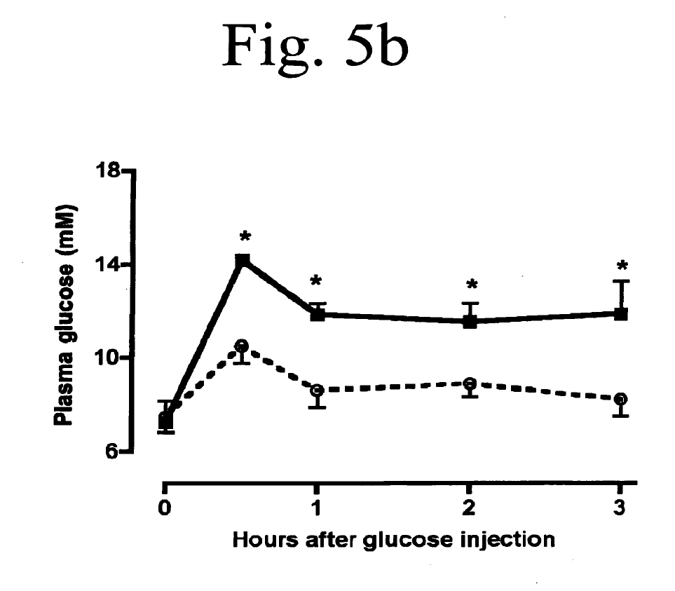

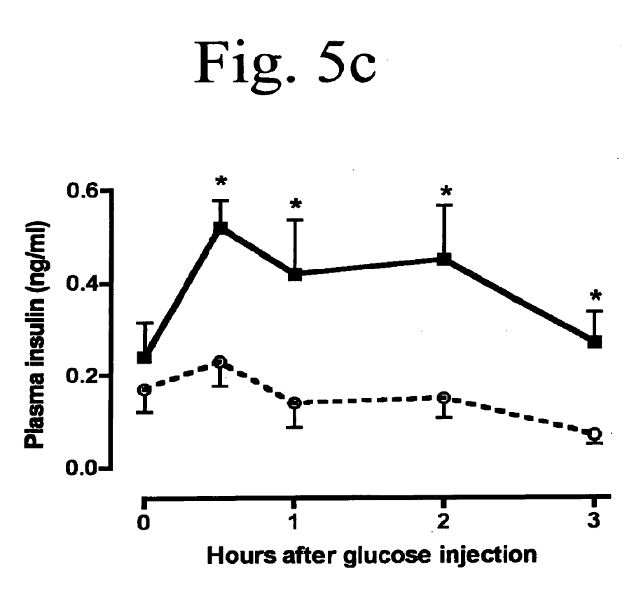

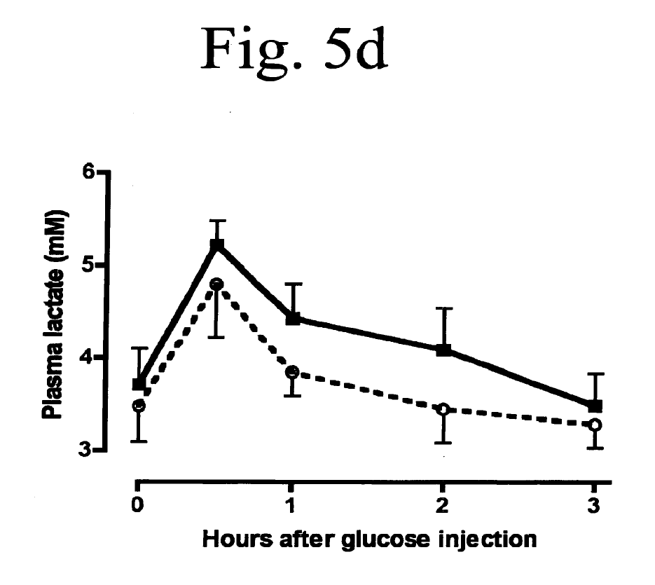

FIGS. 5(a) through 5(d) show standard provocative insulin and glucose tolerance tests, for intraperitoneal insulin tolerance test (ITT) and OGTT response for Pten^{+/+} and Pten^{+/-} mice. FIG. 6(a) illustrates a faster decay of plasma [6,6-²H₂] glucose obtained by the method of the present invention for a mouse model of enhanced insulin action, the Pten heterozygous mouse, reflecting (mainly) enhanced muscle uptake of glucose during the glucose tolerance test. FIG. 6(b) shows the relative rate of de-deuteration of [2-²H₁] vs. [6,6-²H₂] glucose was smaller for Pten heterozygous mice, implying that hepatic phosphorylation of glucose is impaired in the Pten heterozygous deficient mouse. FIG. 6(c) illustrates where de-deuteration of [2-²H₁] vs. [6,6-²H₂] glucose occurs in hepatic metabolic pathways, with use of just the [2-²H₁] and [6,6-²H₂] glucose tolerance test, i.e. the hepatic recycling deuterated glucose tolerance test (HR-dGTT). FIGS. 6(b) shows glucose recycling increases during an OGTT, as measured by the HR-dGTT of the present invention, as discussed in below.

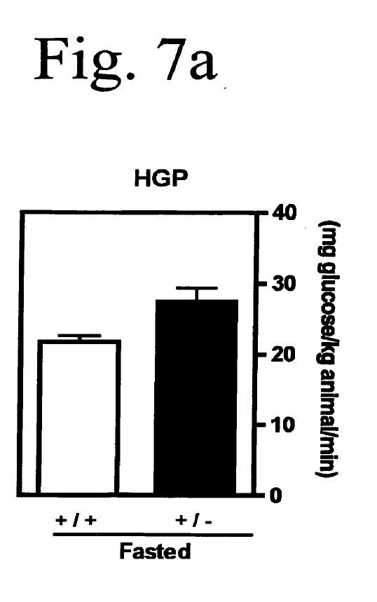

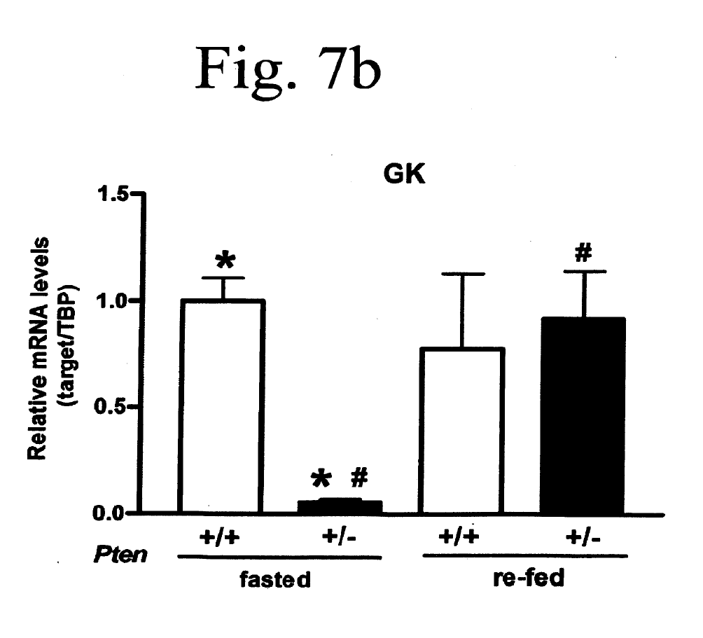

FIGS. 7(a) and 7(b) validate that glucokinase expression for the Pten heterodeficient mouse is dramatically reduced in the fasted state to preserve HGP in the fasted state. The decreased glucoinase expression in the basal state of Pten heterodeficient mice is seen despite evidence for enhanced hepatic insulin action, such as decreased hepatic phosphoenolpyruvate carboxykinase expression in the fasted state, and increased induction of glucokinase between fasted and fed states in Pten heterodeficient mice. This indicates that the measurement of [2-²H₁] versus [6,6-²H₂] glucose recycling are a biomarker for detecting glucose homeostatic changes for the whole body metabolic network, as glucokinase expression is being controlled, probably by a Central Nervous System (CNS) homeostatic mechanism, to insure adequate glucose output in the fasted state for the brain's energy need.

In hepatic cell culture, the literature shows that glucokinase expression increases enormously in response to insulin. In vivo, in the Pten heterodeficient mouse, a model for increased insulin sensitivity, despite increased hepatic insulin sensitivity glucokinase is nearly totally suppressed in the fasted state, even though the Pten heterodeficient mouse has the same glucose and insulin values as the wild type mouse. However, the increased hepatic sensitivity of the Pten is demonstrated in the powerful induction of glucokinase expression between the fasted and fed states of the Pten heterodeficient mouse, an induction not evident for the wild type mouse. The lower value of 1-D1/D2 (glucose recycling, FIG. 6(b)) for the Pten heterodeficient mouse reflects the suppression of glucokinase in the fasted state, as well as the increased induction of glucokinase over the time course of the GTT, in comparison to the wild type. This indicates that glucose recycling (1-D1/D2) is a biomarker of glucose homeostasis as it includes an assessment of CNS control of hepatic glucose metabolism in the fasted state, as well as a measurement of the response to hepatic insulin action in the fasted to fed transition, in this case, during a GTT. Hepatic recycling also is a biomarker for the compensatory response for changes in peripheral insulin sensitivity.

It had been known for some time that T2DM patients, who have decreased peripheral insulin sensitivity and decreased ability to dispose of glucose, have increased hepatic glucose recycling. The interpretation from the DAFST is that this indicates decreased peripheral glucose uptake seen even in early Type II diabetes, compensated for by increased hepatic glucose uptake. Liver glucose metabolism is much more insulin sensitive than the stimulation of glucose uptake by skeletal muscle. Since skeletal muscle mass is much larger than liver mass, the hyperinsulinemia that develops with T2DM can stimulate increased hepatic glucose uptake.

The HR-dGTT of the present invention performed in a model of enhanced insulin sensitivity (Pten heterodeficient mouse) shows that the converse of the T2DM situation was also true; that enhanced peripheral glucose disposal and insulin sensitivity is associated with decreased hepatic glucose recycling, in order to preserve basal (fasting) hepatic glucose production for the brain's use. The brain, even though an insulin insensitive tissue, under normal diurnal feeding conditions uses glucose almost exclusively for energy, and thus one purpose for hepatic glucose recycling is to satisfy the liver's contribution to the brain's energy needs.

In the present invention, use of the HR-dGTT for glucose recycling and peripheral glucose disposal measurements allows the assessment of compensations between the handling of glucose between the liver and muscle. Glucose recycling measurements can also reflect neural control of hepatic glucose production. For example, the lower values of glucose recycling (1-D1/D2) obtained for the Pten heterozygous mouse in comparison to wild type reflect a homeostatic need to preserve fasting plasma glucose, and HOP at levels needed for the brain. See explanations associated with FIGS. 7(a) and 7(b), showing preservation of hepatic glucose production in the Pten heterozygous mouse in the fasted state, resulting from the suppression of the fasting hepatic glucokinase level.

For T2DM diabetics, the increased hepatic glucose phosphorylation and increased hepatic glucose uptake is hypothesized to be a defensive, compensatory mechanism for lowering blood glucose, due to decreased muscle glucose uptake due to insulin resistance. The HR-dGTT provides a way to easily assess fuel compensations between organs that maintain glucose homeostasis, failure of which suggests the pre-diabetic state. HR-dGTT testing in humans supports these results.

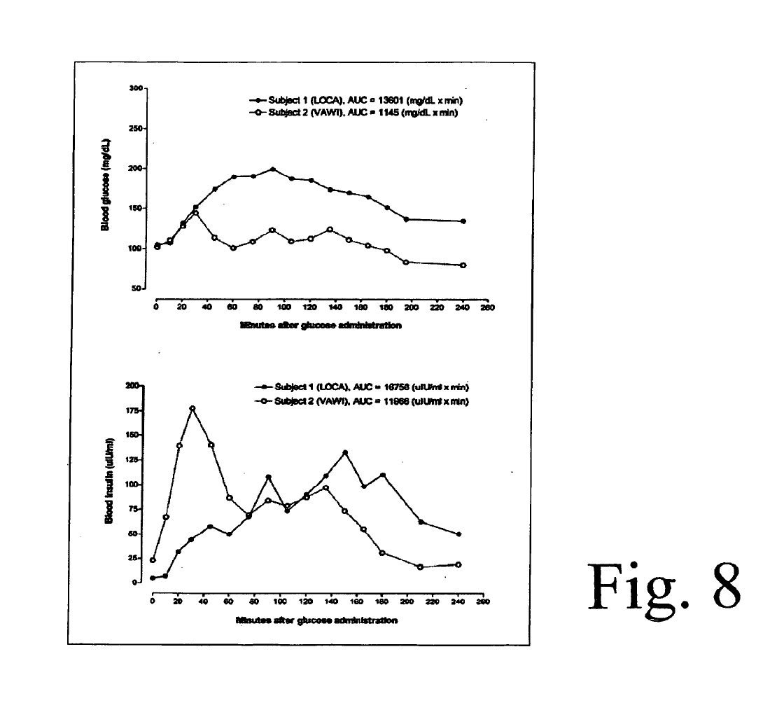

FIG. 8 illustrates the plasma glucose and insulin response during the oral HR-dGTT. In FIG. 8, Subject 1 is far more insulin resistant than Subject 2, as the AUC for the plasma glucose response is 8-fold higher for Subject 1 in comparison to Subject 2 (13601 vs. 1145 mg/dl×min), and the AUC for plasma insulin is approximately 40% higher for Subject 1 compared to Subject 2 (16756 vs. 11966 uU/ml×min). Both Subject 1 and Subject 2 are similarly obese, with a body mass index (BMI) of 33. Notably, the glucose and insulin response of Subject 1 is abnormal, as opposed to the compensated insulin and glucose responses of Subject 2 which result in minimal impaired fasting glucose, and a normal post-pransial glucose response. Analysis of the blood insulin time course shows that the first phase can be determined by analysis of the initial peak portion of an entire Area AUC of blood insulin.

Assessment of the plasma glucose and insulin alone shows Subject 1 to have the American Diabetes Association classification of impaired glucose tolerance, either by two hour post-prandial sugar being above 140 mg/dl, or by fasting glucose being above 100 mg/dl. Subject 1 has also lost the immediate release of insulin (preformed insulin stored in the pancreatic □-cells), as seen that there is a very small insulin response in the 1 hour period after glucose administration. For Subject 1, loss of the first phase insulin release, along with an larger, more extended plasma glucose response, is characteristic of a final developmental stage for T2DM, in view of an extended second phase insulin response between 90 and 240 minutes for Subject 1.

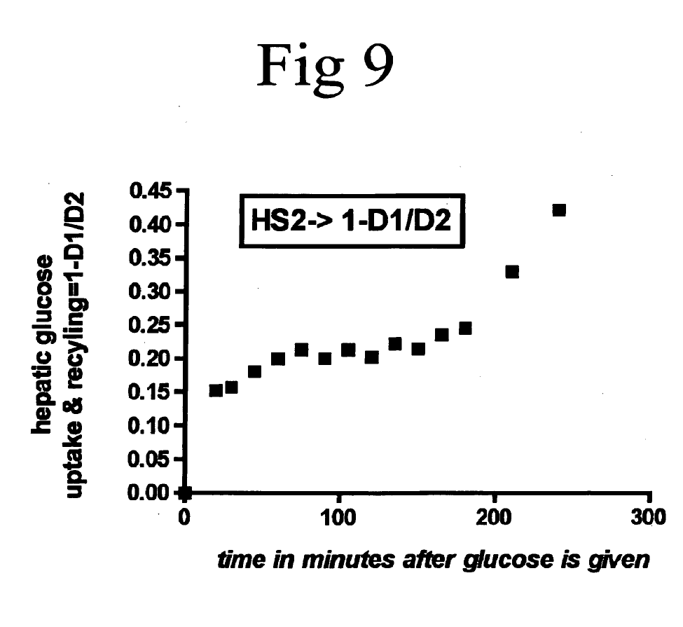

FIG. 9 shows hepatic glucose uptake and recycling for Subject 2, measured by examining a percentage difference in the plasma enrichments between [2-²H₁] glucose (D1) and [6,6-²H₂] glucose (D2) assessed during the entire oral HR-dGTT. Shortly after the oral glucose load, 1-D1/D2=0.15, so D2=1.18*D1. A linear region of change in the difference between D1 and D2 occurs, followed, by a constant region at approximately 100 minutes. At 100 minutes, 1-D1/D2=0.2 for approximately 50 minutes, with D2=1.25*D1. Since equal amounts of D1 and D2 were given, the more rapid decay of D1 relative to D2 indicates hepatic glucose uptake and recycling in the liver. Hepatic recycling results in more rapid loss of [2-²H₁] glucose (D1) and [6,6-²H₂] glucose (D2) seen in plasma, due to export of glucose that preferentially lost its label on the 2^{nd} carbon. See FIG. 6(c), for illustration of the mechanics of the stable isotope decay.

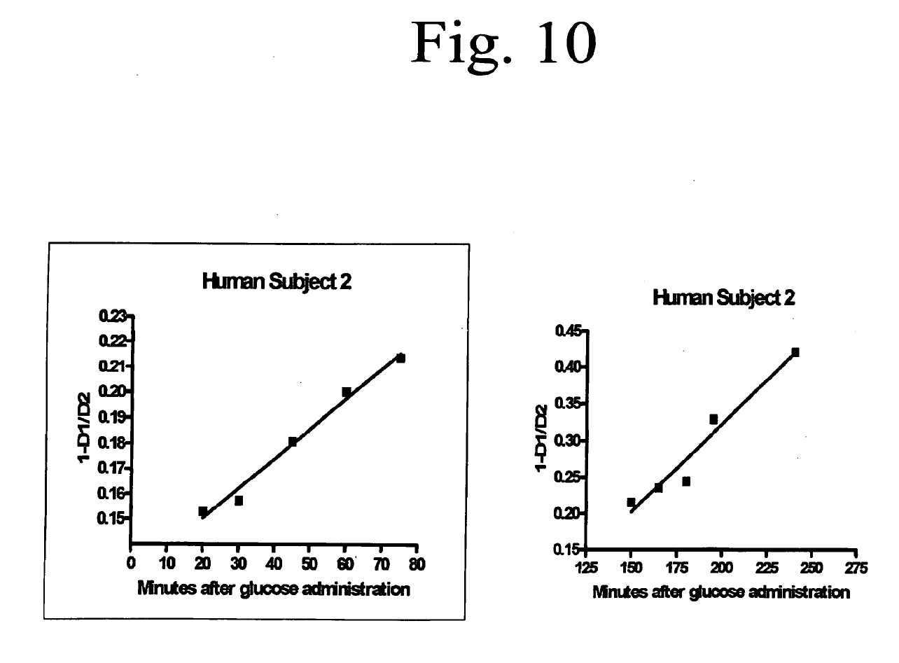

The linear regions of change in the difference between the plasma [2-²H₁] and [6,6-²H₂] glucose enrichments during the oral HR-dGTT for Subject 2 are shown in FIG. 10. These linear regions mainly reflect insulin stimulation of hepatic glucokinase, as [2-²H₁] glucose cannot enter the liver unless it is phosphorylated to glucose-6-phosphate by glucokinase.

In rodents, the 1-D1/D2 response is linear during a glucose tolerance test, and the magnitude of the response is dependent on the induction of hepatic glucokinase during the fasted to fed transition that occurs during a glucose tolerance test. See FIG. 6(b). The increased glucose recycling seen in FIG. 10 for the 150-240 minute interval vs. the 20-75 minute interval after glucose administration (2-fold increased slope of the 1-D1/D2 vs. time response) is attributed to the induction of hepatic glucokinase due to first phase insulin release (between 0 and 75 minutes) becoming evident in this 150-240 minute interval. Examination of the glucose response in FIG. 8 also shows the plasma glucose dipping below the original fasting level during this 150-240 minute interval, probably due to increased glucose uptake by the liver.

FIG. 9 shows hepatic glucose uptake and recycling for Subject 1, measured by examining a percentage difference in the plasma enrichments between [2-²H₁] glucose (D1) and [6,6-²H₂] glucose (D2) assessed during oral HR-dGTT. Shortly after the oral glucose load, 1-D1/D2=0.22, so D2=1.28*D1, and was observed to remain constant throughout the HR-dGTT. The lack of linearity, or change, of 1-D1/D2 over the HR-dGTT, indicates hepatic insulin resistance, and lack of the first phase of insulin release.

A percentage difference in the plasma enrichments of [2-²H₁] glucose (D1) and [6,6-²H₂] glucose (D2) was assessed during the oral HR-dGTT and is shown on the y-axis of FIG. 10. In light of the glucose recycling, and metabolic measurement results for the mouse model of increased insulin sensitivity presented, the results shown in FIG. 10 are interpreted as follows. The linear decay of the percentage difference between the plasma [2-²H₁] and [6,6-²H₂] glucose enrichments during the oral HR-dGTT (glucose recycling/hepatic glucose phosphorylation=the 1-D1/D2 response) for Subject 2 reflects the action of hepatic glucokinase, as the [2-²H₁] glucose cannot enter the liver unless it is phosphorylated to glucose-6-phosphate by glucokinase. In rodents, the 1-D1/D2 response is linear during a glucose tolerance test, and the magnitude of the response is dependent on the basal activity of glucokinase, as well as the induction of hepatic glucokinase during the fasted to fed transition that occurs during a glucose tolerance test. See discussion of Pten heteodeficient mice results in FIGS. 6 and 7. The increased glucose recycling seen in FIG. 10 (right panel) for the 150-240 minute interval compared to the 20-75 minute interval after glucose administration in FIG. 10 (left panel), i.e. a two-fold increase in slope of the 1-D1/D2 vs. time response, is estimated to reflect induction of hepatic glucokinase due to first phase insulin release that occurs between zero and approximately seventy-five minutes, this glucokinase induction becoming evident as increased glucose recycling in the 150-240 minute interval of FIG. 10 (right panel).

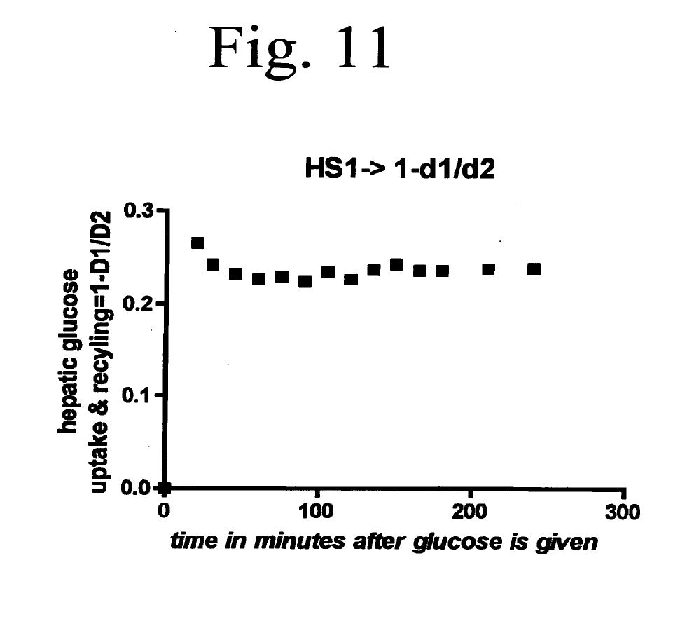

Examination of the glucose response in FIG. 8 also shows plasma glucose dipping below the original fasting level during the 150-240 minute interval, as estimated due to increased glucose uptake by the liver. The 1-D1/D2 response of Subject 1 is flat throughout the entire time course of the HR-dGTT (FIG. 11), reflecting the underlying hepatic metabolic dysregulation of early T2DM. The failure of Subject 1 to have such a linear 1-D1/D2 response reflects the lack of first phase insulin release in Subject 1 (FIG. 8) and thereby the failure to suppress hepatic glucose production, despite the increase in second phase insulin release of HS1 vs. HS2. The insulin level of HS1 may be high for most of the day, despite the low fasting glucose, as it seen from FIG. 8 that at the end of the four hour OGTT, the insulin levels of HS1 are still 10 fold higher than in the fasting state. The chronically high plasma insulin levels may induce a moderately high, fixed level of glucokinase. The failure of Subject 1 to have a linear 1-D1/D2 response therefore may be attributed to the liver trying to compensate for the hyperglycemia, in the face of hyperinsulinemia, by fostering a constant increased uptake of glucose, insensitive to slow changes in second phase insulin release.

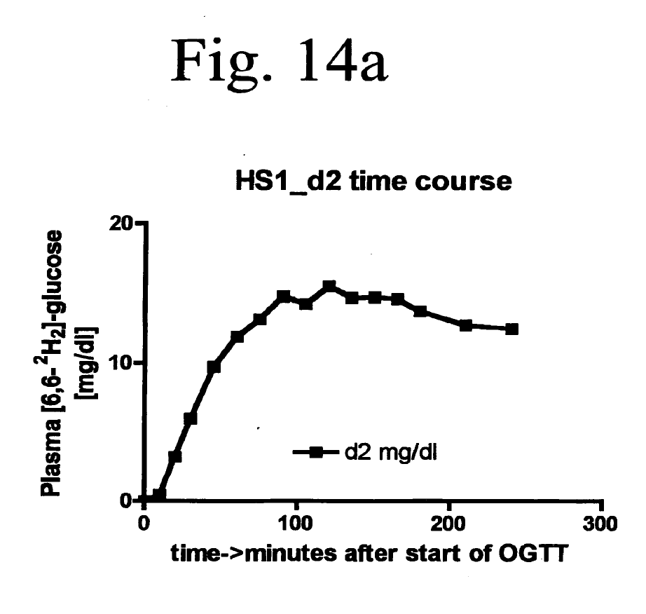

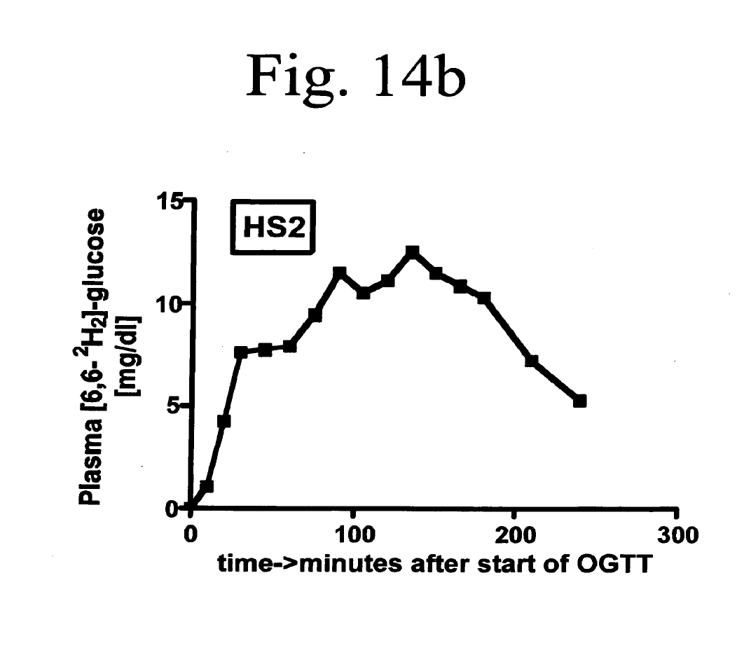

Accordingly, Subject 1 shows a very significant degree of hepatic insulin resistance; along with the increased peripheral insulin resistance indicated by quantification of the plasma glucose and insulin responses to the HR-dGTT (FIG. 8), along with the D2 time course (FIG. 14). For Subject 1, D2 glucose stays elevated relative to that seen for Subject 2, despite a higher insulin AUC than Subject 2, indicating very significant peripheral insulin resistance.

That is, FIGS. 8-11 and 14 illustrate an analysis between a patient with very early, well compensated impaired glucose tolerance (Subject 2), and a patient with late stage, impaired glucose tolerance, early T2DM (Subject 1). Both Subject 1 and Subject 2 can be considered to be types of pre-diabetes, with HS1 being more severe, as having IGT by two hour post-prandial between 140 and 200 mg/dl, as well as fasting glucose being between 100-125 mg/dl. Subject 2 has a very early form of pre-diabetes, as first phase insulin response is sufficient to prevent post-prandial hyperglycemia, with a nearly normal fasting glucose. Subjects 1 and 2 have a dramatically different glucose recycling response (1-D1/D2), with Subject 1 demonstrating a moderately high, constant value of 1-D1/D2 thought to reflect high average (over a given day) insulin secretion, along with hepatic insulin resistance. The glucose recycling response (1-D1/D2) of Subject 2 differs, in having inducible linear regions, which seem to be responsive to the levels of insulin secreted during the first phase. (See FIGS. 9 and 10).

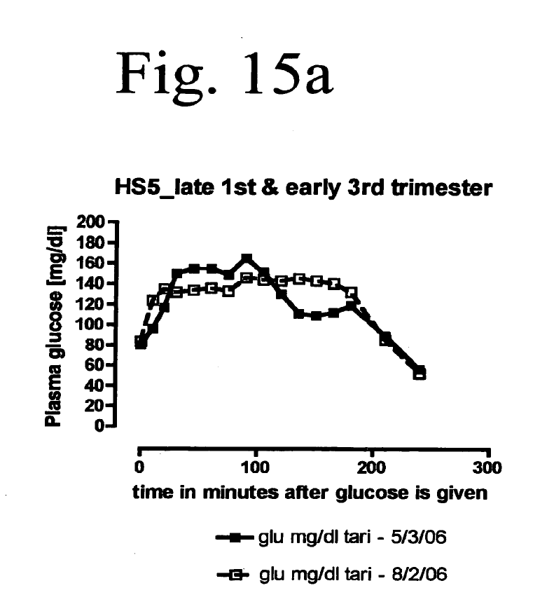

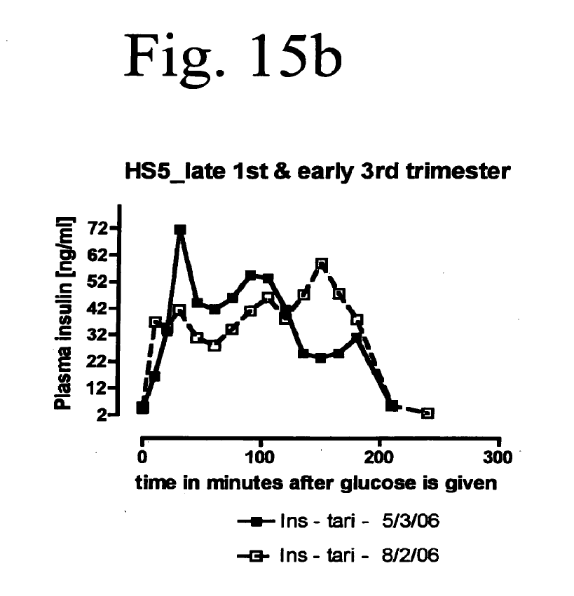

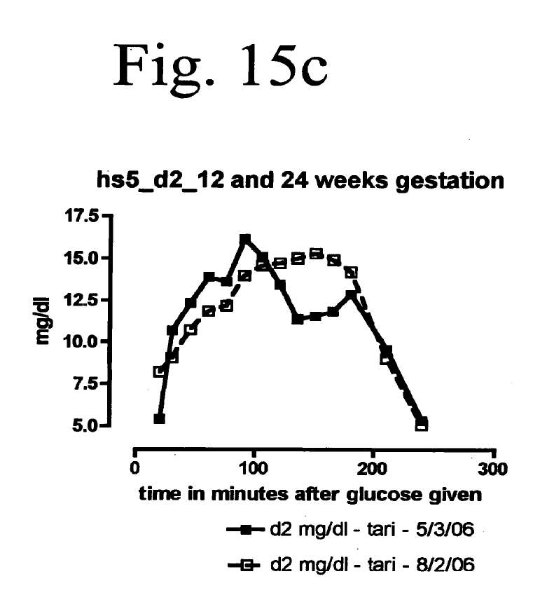

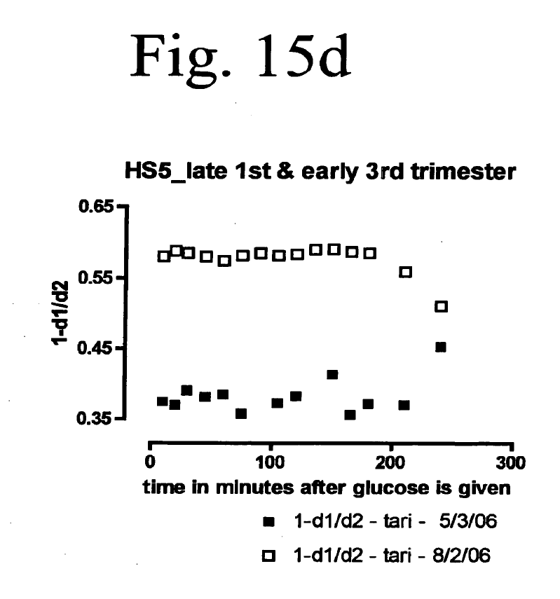

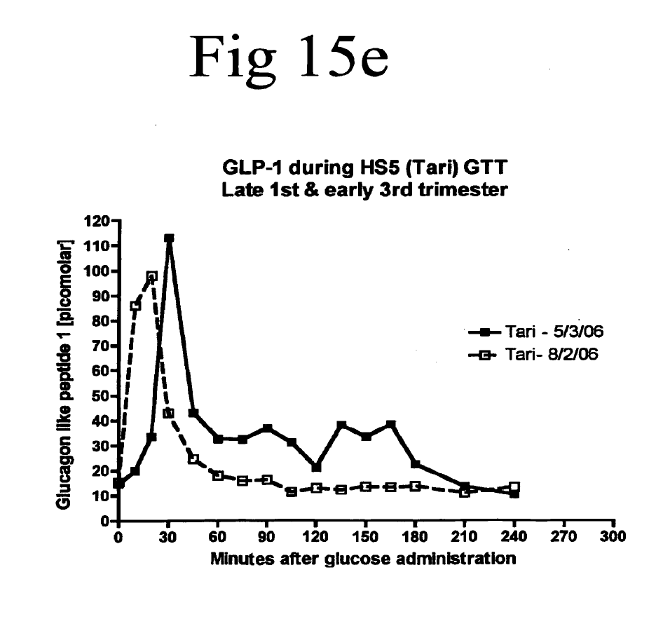

To illustrate the utility of the DAFST for examining very early, reversible, changes in hepatic and peripheral insulin resistance, normal progression of hepatic and peripheral insulin resistance were examined during pregnancy using the HR-dGTT. As shown in FIG. 15, the patient exhibited a normal fasting and post-prandial glucose response in response to the 100 gram OGTT at the end of the patient's first trimester and the beginning of the third trimester. However, there was an approximate 50% reduction in the magnitude of the first phase insulin release, when comparing the insulin response to an OGTT at the beginning of the third trimester, to that of the end of the first trimester. The glucose recycling response (1-D1/D2) was also dramatically different for the end of the first trimester vs. the beginning of the third trimester, with the glucose recycling response at the end of the first trimester appearing as the very early form of pre-diabetes seen for patient HS2 (FIGS. 9 and 10), and the glucose recycling response at the beginning of the third trimester appearing as the late form of pre-diabetes seen in subject HS1 (FIG. 11). In the first trimester there is a linear 1-D1/D2 time response during the HR-dGTT between 90 and 150 minutes, and between 165 and 240 minutes. This is similar to the 1-D1/D2 response of subject HS2 (see FIGS. 9 and 10). The 1-D1/D2 biomarker can signify loss/resistance to hormonal mechanisms regulating first phase insulin release. FIG. 15(e) shows that the GLP-1 response in the first trimester (during the HR-dGTT) is sharper, higher and more prolonged than seen in the third trimester, and it is known that GLP-1, under conditions of insulin resistance, can also be associated with less of an effect to stimulate first phase insulin secretion. Accordingly, the DAFST provides a valuable predictor of pre-diabetes, and is also of great utility for the diagnosis of glucose homeostasis changes that could result in gestational diabetes.

The HR-dGTT is the part of the preferred DAFST that assesses glucose homeostasis, and control/feedback mechanisms. It will be recognized that other stable isotopes of glucose can be used instead of deuterated glucose for the DAFST, and each unique glucose label will give a different view of metabolic pathways that utilize glucose, and affect fuel switching. For example, it has been shown in mouse studies (See, Determination of a glucose dependent futile re-cycling rate constant from an IPGTT, X-Xu, J. Xiao, G., Trujillo, C., Chang, V., Blanco, L., Chung, B., Makabi, S., Saad, M., Ahmed, S., Bassilian, S., Lee, W. N. P. and Kurland, I. J., Analytical Biochemistry 315: 238-246, 2003; Peroxisomal Proliferator-Activated Receptor alpha Deficiency Diminishes Insulin-Responsiveness of Gluconeogenic/Glycolytic/Pentose Gene Expression and Substrate Cycle Flux, Xu, J., Chang, V., Joseph, S. B., Bassilian, S., Saad, M. F. Lee, W. N. P. and Kurland, I. J, Endocrinology 145(3):1087-95, 2004, the contents of which is incorporated herein by reference) that [1,2-¹³C] glucose can also be used as for a hepatic recycling glucose tolerance test (HR-1,2¹³C-GTT). The HR-1,2¹³C-GTT yields parameters that reflect activity of glucose recycling through the hepatic pentose and tricarboxylic acid (TCA)/Cori cycles. Cori cycling refers to the substrate cycle in which glucose produced by the liver is added to that in plasma, and circulates to peripheral tissues, which converts the glucose to lactate (skeletal muscle is the major contributor). The lactate produced then re-circulates back to the liver and forms glucose again, completing a substrate cycle between the liver and peripheral tissues (mainly skeletal muscle, due to its mass) that involves the conversion of glucose to lactate and back again. Dysregulation in Cori cycling can be uniquely assessed using either [1,2-¹³C] glucose or [1,6-¹³C] glucose. [1,6-¹³C] glucose cannot be used to assess hepatic pentose pathway recycling, as [1,2-¹³C] glucose can. The hepatic pentose cycle helps regulate hepatic carbohydrate usage, and the conversion of carbohydrates to fatty acids.

Accordingly, utility exists is using different glucose labels for different HR-dGTTs as part of the DAFST, as each label used can separate out a different, or overlapping, portion of metabolic pathways in vivo. In addition, while administration of carbohydrates as a liquid glucose solution has been demonstrated, the labeled glucose can also be administered as part of a mixed meal, and the DAFST can assess dysregulation of fuel switching before and after the administration of a mixed fuel meal, which contains glucose, fats and protein. See, Determination of a glucose dependent futile re-cycling rate constant from an IPGTT, X-Xu, J. Xiao, G., Trujillo, C., Chang, V., Blanco, L., Chung, B., Makabi, S., Saad, M., Ahmed, S., Bassilian, S., Lee, W. N. P. and Kurland, I. J., Analytical Biochemistry 315: 238-246, 2003; Peroxisomal Proliferator-Activated Receptor alpha Deficiency Diminishes Insulin-Responsiveness of Gluconeogenic/Glycolytic/Pentose Gene Expression and Substrate Cycle Flux, Xu, J., Chang, V., Joseph, S. B., Bassilian, S., Saad, M. P. Lee, W. N. P. and Kurland, I. J, Endocrinology 145(3):1087-95, 2004; and Pub No. US 2005-0238581 A1 (U.S. patent application Ser. No. 11/060,640), and Pub No. US 2005/0281745 A1 (U.S. patent application Ser. No. 11/184,546), which are incorporated herein by reference.

The ability of the DAFST to assess switching between glucose and fatty acids is seen by comparing the insulin and glucose response to the time course of plasma acetyl-carnitine, and plasma fatty acyl-carnitines, measured at 0, 1, 2, 3 and 4 hours during the HR-dGTT protocol. The acyl-carnitines are a group of metabolites that can be derived from mitochondrial fatty acyl-coenzyme A intermediates, formed during fatty acid oxidation by the carnitine acyl transferases. These acyl-carnitine fatty acid intermediates are vital to the transport of fatty acids across the mitochondrial membrane. By using liquid chromatography/mass spectrometry these acyl-carnitine fatty acid intermediates can be measured in plasma. The relevance of measuring these intermediates to assess defects in fatty acid fuel utilization stems from understanding the function of the acyl-carnitine fatty acid intermediates. If fatty acid utilization is blocked, the activated form of fatty acids, the fatty acid Coenzyme As (fatty acyl CoAs), would accumulate, and the lack of available CoA would limit carbohydrate metabolism. The exchange of Coenzyme A for carnitine allows the transport of excess fatty acids that cannot be metabolized out of the mitochondria, so in essence, measurement of fatty acyl-carnitines in plasma allows the ability to assess, non-invasively, measurement of fatty acid oxidation intermediates, or acetyl CoA, levels in mitochondria.

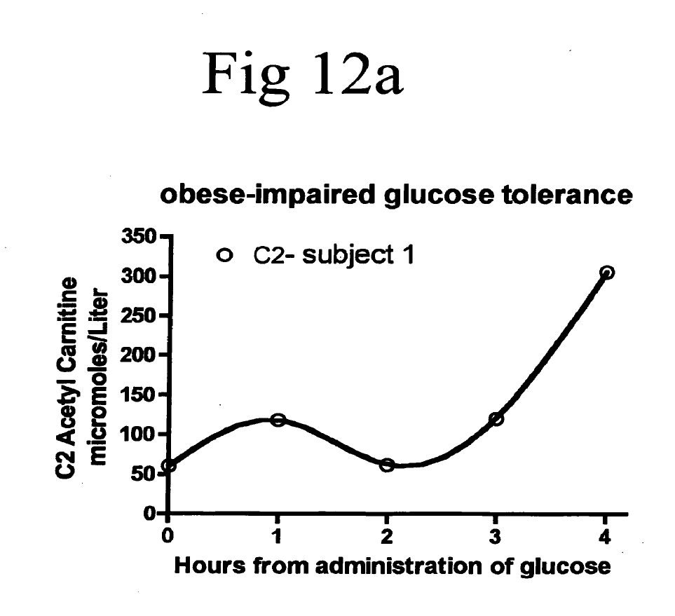

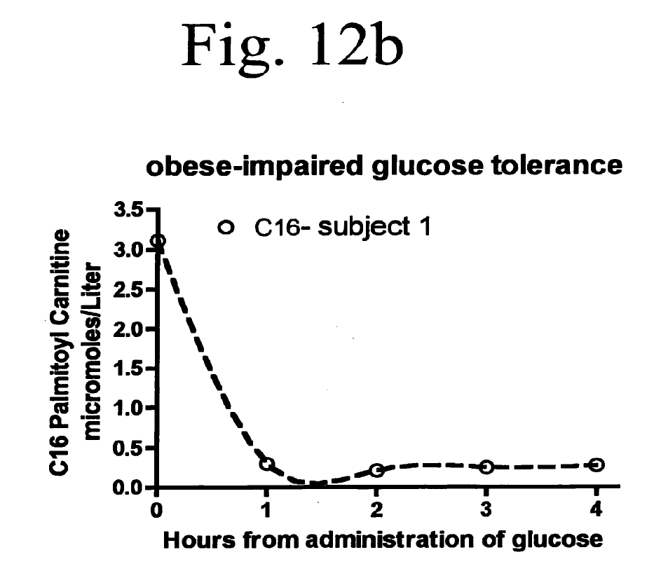

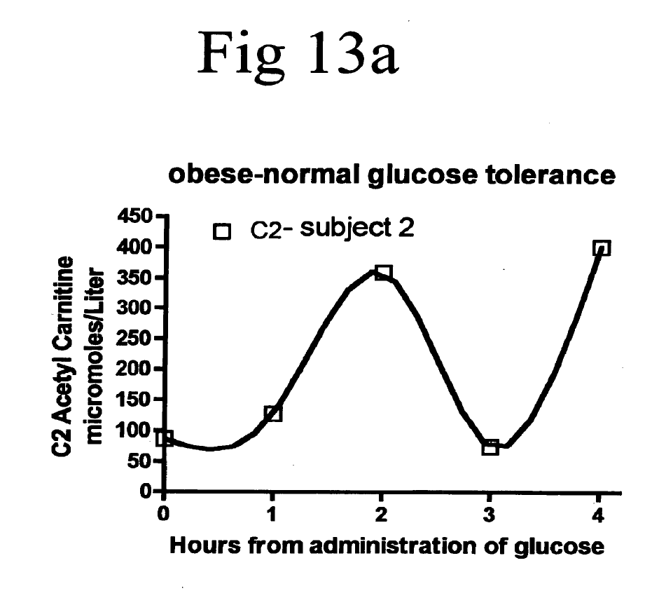

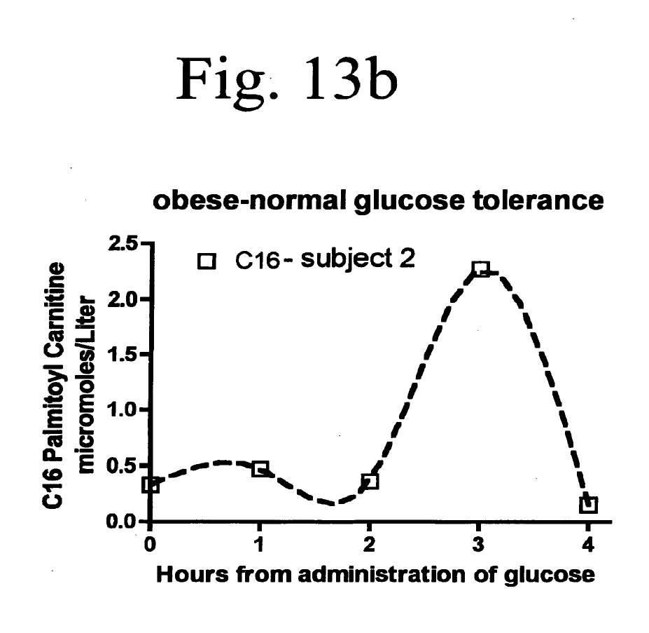

FIGS. 12(a)-(b), and 13(a)-(b) show plasma acetyl-carnitine (C2), and plasma palmitoyl-carnitine (C16) measured during the oral hepatic recycling deuterated glucose tolerance test (HR-dGTT) of Subject 1 and Subject 2, respectively. FIGS. 12(a) and 12(b) are the plasma acetyl-carnitine and palmitoyl-carnitine levels measured for Subject 1, an obese female patient with severely impaired glucose tolerance, as discussed in regard to FIGS. 8, 11 and 14. FIGS. 13(a) and 13(b) are the plasma acetyl-carnitine and palmitoyl-carnitine levels measured for Subject 2, an obese female patient with minimally impaired glucose tolerance. These acetyl-carnitine and palmitoyl carnitine levels can be interpreted with importance to fuel switching when compared to glucose, insulin and leptin time courses seen during the HR-dGTT.

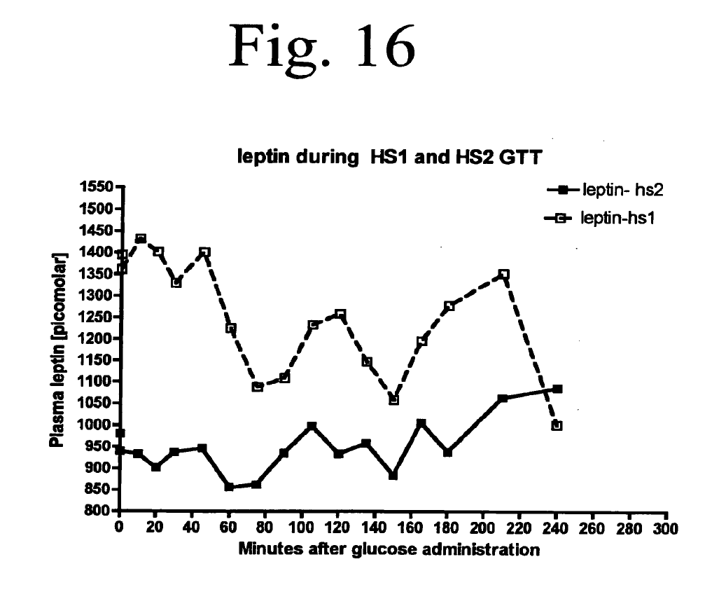

As can be seen from FIG. 8, plasma glucose peaks at ninety minutes for Subject 1, but the curve is very broad, a function of the lack of first phase insulin release for Subject 1. Plasma glucose is still not back to baseline levels by 240 minutes. Subject 1 has an insulin peak at 160 minutes, but the peak is broad, and higher than that of Subject 2. Subject 1 also has high plasma leptin levels throughout the HR-dGTT. (See FIG. 16). Subject 2 has a sharp glucose peak at 30 minutes, and glucose is back to baseline values by 60 minutes. Subject 2 has a high, sharp first phase insulin release, which peaks to 5 times basal at 30 minutes, and is still two times basal at 80 minutes, which is where the second phase insulin response is seen to begin, peaking at 140 minutes before dropping back to basal by 210 minutes. It is notable that the plasma leptin response is much lower for Subject 2 than for Subject 1 throughout nearly the entire HR-dGTT. (See FIG. 16.)

FIGS. 9 and 10 show that hepatic glucose disposal for Subject 2 was accelerated between three and four hours, attributed mainly to the induction of glucokinase caused by first phase insulin release. Subject 2 has normal glucose tolerance, achieved by adaptive secretion of insulin, as she has a high basal level of insulin (25 microunits/ml) and a very high peak first phase of insulin (175 microunits/ml). While the high levels of insulin suggest she has insulin resistance, the normal time secretion pattern of glucose and insulin disposal indicate that she has compensated for insulin resistance with insulin secretion, has normal glucose tolerance, and can handle fuel switching normally. This is indicated also from the plasma acyl-carnitine time courses. In the fasted state, the low plasma palmitoyl-carnitine levels indicate she is burning fatty acids well, so there are only low levels of fatty acyl-carnitine that can diffuse from mitochondria into plasma. Plasma palmitoyl-carnitine levels peak at 3 hours, where Subject 2 is having increased hepatic glucose disposal, and in general, humans use glucose at this point in the fed state after meals as a normal physiological response. The drop in plasma palmitoyl carnitine levels is consistent with the time course of the drop in plasma insulin levels, which are seen to drop steadily for Subject 2 between 140 and 210 minutes. The low plasma insulin values allow plasma fatty acids to be burned, and it can be seen that plasma palmitoyl carnitine levels drop as a consequence in Subject 2. Acetyl carnitine peaks twice during the HG-dGTT for subject 2, once at 2 hours, where it reflects increases in acetyl CoA due to glycolysis, and once at 4 hours, where it reflects acetyl CoA due mainly to fatty acid oxidation, as reinforced by the time course of palmitoyl-carnitine discussed for Subject 2.

The impairment in glucose tolerance seen for Subject 1 reflects a more general disorder in fuel switching, as is very evident from examination of the acyl carnitine time course, especially in light of the leptin response. The high palmitoyl-carnitine at time zero in the fasted state reflects metabolic inflexibility. Metabolically inflexible patients inappropriately metabolize more glucose than normal in the fasted state, and metabolize more fatty acids than normal in the fed state, after meals. The high levels of blood palmitoyl carnitine in Subject 1 in the fasting state indicates that fatty oxidation is impaired. Leptin fosters fatty acid oxidation, and the impairment of palmitate oxidation seen for Subject 1, in the presence of high leptin levels, indicates a state of leptin resistance, seen in Type II DM. Measurement of plasma acyl carnitines during the HR-dGTT as part of the DAFST yields a biomarker not only for metabolic inflexibility, but as a biomarker for the underlying impaired hormonal effects resulting in metabolic inflexibility. The high palmitoyl-carnitine in the fasted state drops quickly after glucose ingestion, despite the lack of first phase insulin release. The acetyl-carnitine level has a small peak at one hour and a large peak at four hours. The acetyl-carnitine peak at 1 hour may reflect mainly the high glucose peak, which begins at one hour for Subject 1, and is smaller than that seen for Subject 2. The second acetyl carnitine peak at four hours again reflects the oxidation of fatty acids when glucose and insulin have come closer to their fasted values.

While the human studies are described of the DAFST is the response of 2 acyl-carnitines, acetyl-carnitine and palmitoyl-carnitine, the LC/MS methodology of the present invention yields approximately three dozen acyl-carnitines presenting having uses in describing the metabolic flexibility of the individual/organism assessed. The above description discusses 2 acyl carnitines in the DAFST patient-acetyl-carnitine and palmitoyl-carnitine, in which an LC/MS run yields approximately a dozen and a half acyl-carnitines, and other runs will be recognized as also proving useful, such as C8 carnitine, for example, though apparently not as dramatic as the C2 and C16 carnitines discussed above.

In a preferred embodiment of the present invention, the DAFST can assess fuel switching between glucose and fatty acid during the transition between the fasted and fed states, using a GTT or meal fed protocol, defining the metabolic flexibility of an individual.

In a preferred embodiment of the present invention, an apparatus performs the DAFST, wherein the apparatus performs a hepatic recycling deuterated glucose tolerance test (HR-dGTT), and analyzes a time course of acyl-carnitines during the HR-dGTT, in particular, acetyl-carnitine and palmitoyl carnitine. In a preferred embodiment of the present invention, the HR-dGTT portion of the DAFST distinguishes and classifies disorders in hepatic versus peripheral (mainly muscle) glucose disposal.

For the DAFST of the present invention, it is preferable not to limit the use of glucose stable isotopes to deuterium labeled 2-deuterated and 6,6 deuterated glucose. There are unique aspects of using 1,2-13C glucose instead of deuterated glucose, and 1,6-13C glucose also has potential uses instead, along with other glucose labels, with the data provided herein as an example of deuterium labeled glucose. Examples are provided of a 100 mg glucose tolerance test, though less may be used, like a 75 gram test of unlabeled glucose, and those of skill in the art can utilize the disclosure herein to utilize more in other embodiments of the present invention.

The above description addresses 2 acyl carnitines in the DAFST patient-acetyl-carnitine and palmitoyl-carnitine (the LC/MS run yields approximately one and a half dozen acyl-carnitines), and others, such C8 carnitine, while not as dramatic as the C2 and C16 carnitines discussed, are expected to be useful.

In addition, further to the administration of carbohydrate as a liquid glucose solution that has been demonstrated, the labeled glucose can also be administered as part of a mixed meal, and the DAFST can assess dysregulation of fuel switching before and after the administration of a mixed fuel meal containing glucose, fats and protein. In a preferred embodiment of the present invention knowing or treating a pre-diabetic state would make a difference in the onset of cardiovascular (macrovascular), or kidney or eye (microvascular) complications.

While the invention has been shown and described with reference to certain exemplary embodiments of the present invention thereof, it will be understood by those skilled in the art that various changes in form and details may be made therein without departing from the spirit and scope of the present invention as defined by the appended claims and equivalents thereof.

What is claimed:

1. A method for measuring and determining whole body fuel homeostasis compensation, the method comprising:
administering a plurality of physiologically acceptable differentially labeled carbohydrates, wherein a first administered carbohydrate label metabolizes faster than at least one of other administered carbohydrate labels;
monitoring recycling rates of at least two of the plurality of labeled carbohydrates; and
detecting efficacy of first and subsequent insulin release phases, wherein the first insulin release phase is detected by comparison of a recycling rate of the first administered carbohydrate with a recycling rate of the subsequent administered carbohydrate label.

2. The method of claim 1, wherein the first phase is defined by analysis of an initial peak portion of an Area Under Curve (AUC) of blood insulin.

3. The method of claim 2, wherein the AUC is between a first time of administration of the plurality of physiologically acceptable differentially labeled carbohydrates and a second time when blood insulin level returns to a level of the first time.

4. The method of claim 3, wherein the first phase occurs within half of an entire AUC time.

5. The method of claim 4, wherein the monitoring step is performed by a plurality of mass spectrometric assessments.

6. The method of claim 1, wherein mass spectrometric monitoring assesses insulin action and fatty acid utilization, as well as effects of other hormonal and metabolic feedback, in response to fasting and fed states.
